(19) United States
(12) Patent Application Publication
(10) Pub. No.: US 2006/0052798 A1
(43) Pub. Date: Mar. 9, 2006

(54) Wire for removing intravascular foreign body and medical instrument

(75) Inventor: Takeshi Kanamaru, Ashigarakami-gun (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Terumo Kabushiki Kaisha, Tokyo (JP)

(21) Appl. No.: 11/219,880

(22) Filed: Sep. 7, 2005

(30) Foreign Application Priority Data
Sep. 7, 2004 (JP) 2004-260097

(51) Int. Cl. A61B 17/26

(52) U.S. Cl. 606/113

(57) ABSTRACT

A wire for removing an intravascular foreign body includes a flexible long wire body, a capturing part which is arranged on the forward end of the wire body and in which is formed a space for capturing an intravascular foreign body, and an auxiliary wire which is arranged on the forward end of the wire body. The auxiliary wire assumes a first state in which it projects into the space and a second state in which it retreats from the space as it moves relative to the capturing part.

This application is based on and claims priority under 35 U.S.C. § 119 with respect to Japanese Application No. 2004-260097 filed on Sep. 7, 2004, the entire content of which is incorporated herein by reference.

### BACKGROUND DISCUSSION

The present invention relates to a device adapted to be used in a medical procedure. More particularly, the present invention pertains to a wire for removing an intravascular foreign body (such as embolus in a vessel) and a medical instrument.

The vital statistics of population published by the Ministry of Health, Labor, and Welfare indicates that cancer dominates in the cause of Japanese death, while heart disease and cerebral apoplexy are the second and third leading causes of Japanese death. The increasing deaths and sequelae due to cerebral apoplexy urgently demand establishment of a therapeutic method.

A recent development in therapy of cerebral apoplexy is thrombolysis which employs a thrombolytic agent to cure brain infarction in its acute phase. It is effective, but its effectiveness is limited. That is, the thrombolytic agent takes a long time for thrombolysis or produces smaller thrombi that scatter to form new emboli. In addition, it has been found that some emboli are insoluble by treatment with a thrombolytic agent.

It has been proved in the U.S. and Europe that the probability to save lives and reduce sequelae would be high if blood flow is resumed within 3 hours after the onset of cerebral apoplexy. Thus, there is a strong demand for development of a new medical instrument that can be inserted into a cerebral vessel to remove the thrombus directly. An example of such a medical instrument is disclosed in Japanese Patent Laid-open No. 2004-16668 which describes a wire for removing an intravascular foreign body.

The wire for removing an intravascular foreign body consists of a wire proper, two branch wires (which branch out from the wire proper), and a plurality of filaments connecting the branch wires. The branch wires and the filaments form a space in which an intravascular foreign body is captured.

The disadvantage of this disclosed wire for removing an intravascular foreign body is that, depending on the size, the wire is sometimes not able to capture an intravascular foreign body.

For example, if the intravascular foreign body is smaller than the space in which it is to be captured, it might slip off through the gap between the filaments. In this case, it would be necessary to exchange the wire for removing an intravascular foreign body that matches the size of the foreign body to be captured. This is undesirably troublesome.

### SUMMARY

The present invention provides a device or wire that is able to more reliably capture and remove an intravascular foreign body. The device or wire for removing an intravascular foreign body includes a flexible elongated wire body, a capturing part arranged at the forward end of the wire body and in which is formed a space for capturing an intravascular foreign body, and an auxiliary wire which is arranged at the forward end of the wire body. The auxiliary wire is movable relative to the capturing part to position the auxiliary wire in a first state in which the auxiliary wire projects into the space of the capturing part and a second state in which the auxiliary wire is retreated from the first state.

The present invention also provides a medical instrument which includes the wire for removing an intravascular foreign body as above-described and a catheter having a lumen for receiving therein the wire for removing an intravascular foreign body.

According to another aspect, a wire for removing an intravascular foreign body comprises a flexible wire tube possessing an outer diameter which permits the flexible wire tube to be positioned in a blood vessel of a living body, a plurality of capturing wires extending forward of a forward end of the flexible wire tube and together defining a space of a size to capture an intravascular foreign body, and an auxiliary wire. The auxiliary wire and the plurality of capturing wires are movable relative to one another to position the auxiliary wire in one position in which the auxiliary wire projects forward of the forward end of the flexible wire tube into the space to engage an intravascular foreign body captured in the space and another position in which the auxiliary wire is retracted relative to the one position in a direction away from the space.

Another aspect pertains to a method of removing an intravascular foreign body from a blood vessel of a living body. The method comprises introducing an intravascular foreign body removing wire in a blood vessel of a living body, with the intravascular foreign body removing wire comprising a flexible elongated wire body, a capturing part positioned forward of a forward end of the wire body, and an auxiliary wire. The method also comprises capturing an intravascular foreign body within a space of the capturing part, initiating relative movement between the capturing part and the auxiliary wire to move the auxiliary wire into engagement with the intravascular foreign body captured within the space of the capturing part, and withdrawing the intravascular foreign body removing wire from the blood vessel with the intravascular foreign body captured within the space of the capturing part.

The wire, instrument and method disclosed herein make it possible to more reliably remove an intravascular foreign body owing to the auxiliary wire that helps capture the foreign body.

### BRIEF DESCRIPTION OF THE DRAWING FIGURES

FIG. 1 is a plan view, partially in longitudinal cross-section, of the wire for removing an intravascular foreign body, in the second state, according to an embodiment of the present invention.

FIG. 2 is a plan view, partially in longitudinal cross-section, of the wire shown in FIG. 1 in the first state.

FIG. 3 is a side view of the wire for removing an intravascular foreign body which is shown in FIG. 1.

FIG. 4 is a schematic diagram illustrating a manner of using the wire for removing an intravascular foreign body which is shown in FIG. 1.

FIG. 5 is a schematic diagram illustrating a manner of using the wire for removing an intravascular foreign body which is shown in FIG. 1.

FIG. 6 is a schematic diagram illustrating a manner of using the wire for removing an intravascular foreign body which is shown in FIG. 1.

FIG. 7 is a schematic diagram illustrating a manner of using the wire for removing an intravascular foreign body which is shown in FIG. 1.

FIG. 8 is a schematic diagram illustrating a manner of using the wire for removing an intravascular foreign body which is shown in FIG. 1.

FIG. 9 is a plan view, partially in longitudinal cross-section, showing the vicinity of the capturing part of the wire for removing an intravascular foreign body (in the first state) according to a second embodiment of the present invention.

FIG. 10 is a plan view, partially in longitudinal cross-section, showing the vicinity of the capturing part of the wire for removing an intravascular foreign body (in the first state) according to a third embodiment of the present invention.

FIG. 11 is a plan view, partially in longitudinal cross-section, showing the vicinity of the capturing part of the wire for removing an intravascular foreign body (in the second state) according to the third embodiment of the present invention.

FIG. 12 is a side view showing the wire for removing an intravascular foreign body which is shown in FIG. 11.

### DETAILED DESCRIPTION

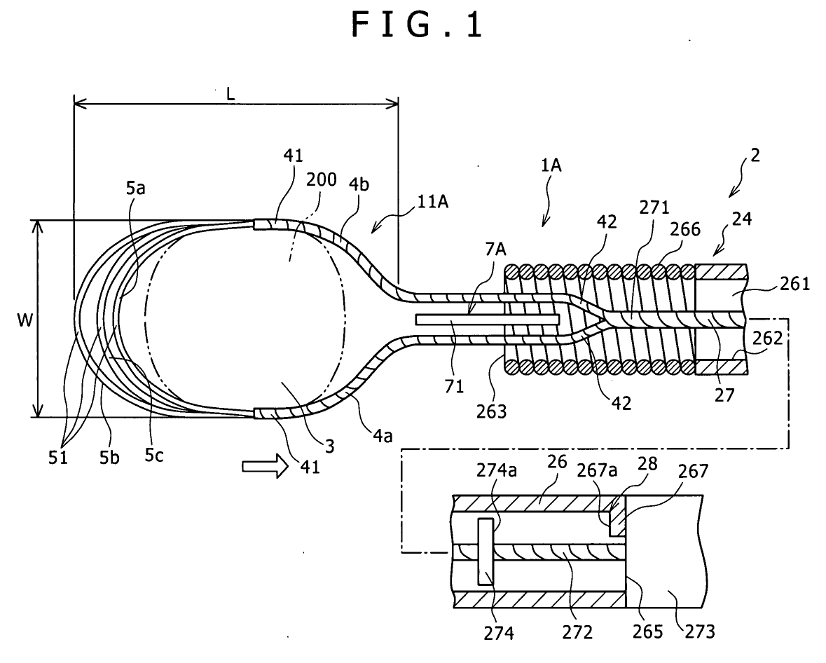

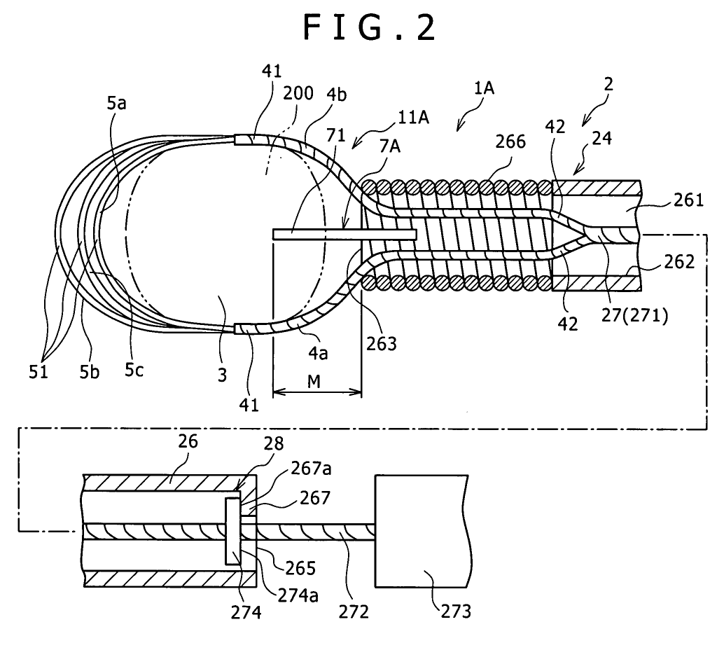

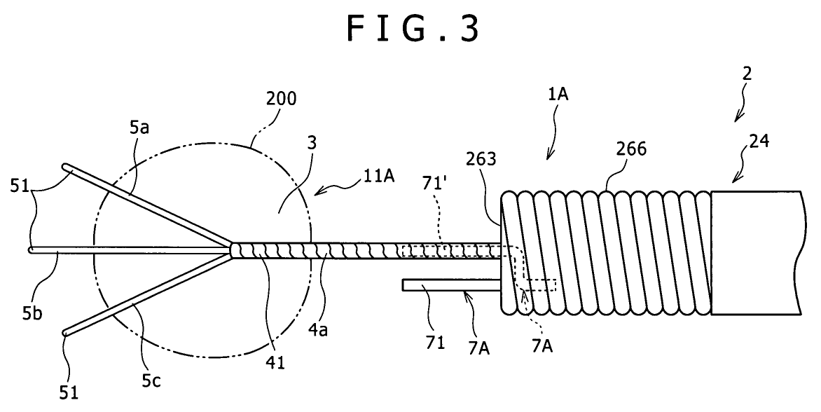

FIGS. 1 and 2 are plan views, partially in longitudinal cross-section, of a wire to remove an intravascular foreign body, illustrating the wire in a second state and a first state respectively, while FIG. 3 is a side view of the wire shown in FIG. 1 and FIGS. 4-8 illustrate how to use the wire shown in FIG. 1 for removing an intravascular foreign body which is shown in FIG. 1. In the following description the right side and left side of FIGS. 1 to 8 are designated as “base end (proximal end)” and “forward end (distal end)”, respectively.

FIG. 1 shows the wire for removing an intravascular foreign body (named 1A), which is intended to capture (or pinch) and remove a foreign body (such as a thrombus and a clot) which can cause an embolism in a vessel 100. A foreign body will be referred to as “embolus 200” hereinafter.

The wire 1A for removing an intravascular foreign body includes a long or elongated wire body 2, a capturing part 11A arranged at the forward end of the wire body 2, an auxiliary wire 7A arranged at the forward end of the wire body 2, and a means 28 for limiting the maximum extent to which the auxiliary wire 7A projects.

The wire body 2 shown in FIG. 1 is comprised of a tube 26 and a linear body 27 which passes through or extends through the inside 261 of the tube 26. The wire body 2 has adequate rigidity and resilience (flexibility) over its entire length.

A coil 266 is provided at the forward end of the tube 26. The coil 266 makes the forward end of the tube 26 (including the coil 266) more resilient or flexible. The tube 26 also possesses a base-end opening 265.

As shown in FIGS. 1 and 2, the linear body 27 is constructed such that it is movable relative to the tube 26. The linear body 27 possesses a base 272 that is capable of projecting from the base-end opening 265 of the tube 26 as shown in FIG. 2.

A manipulating member 273 is provided at the base end 272 of the linear body 27. The manipulating member 273 is used to manipulate or move the linear body 27 in its lengthwise direction. The manipulating member 273 also permits the linear body 27 to be grasped easily at the time of its handling.

The parts forming the wire body 2 may be formed from any materials without specific restrictions, such as metallic and plastic materials, which may be used alone or in combination.

The wire body 2 may vary in length depending on the position and size of the vessel 100 to which it is to be applied. A preferred length ranges from 500 to 4000 mm usually, more preferably 1500 to 2200 mm.

The wire body 2 (or the tube 26) may also vary in thickness (outside diameter) depending on the position and size of the vessel 100 to which it is to be applied. A preferred outside diameter is usually 0.1 to 2.0 mm on average, more preferably 0.25 to 0.9 mm.

The wire body 2 (or the tube 26) should preferably be comprised of a first part which is comparatively hard and located at the base end, a third part which is comparatively soft and located at the forward end, and a second part which is variable in flexibility and positioned at an intermediate position between the first part and third part. In other words, the wire body 2 should preferably be formed in such a way that it gradually decreases in rigidity (flexural and torsional rigidity) from its base end to its forward end. The gradually changing rigidity permits manual manipulation to be reliably transmitted to the forward end 24 (including the coil 266) of the wire body 2. With properties making the forward end 24 relatively flexible, the wire body 2 easily proceeds and bends in the vessel 100 without damaging the vessel 100. Such properties also permit the wire body 2 to maintain its torque transmission and its pushability while inhibiting or preventing kink resistance (or flexing). This contributes to higher safety.

The wire body 2 (or the tube 26) may have a coating layer on its outer surface for reducing friction with the inside of the catheter 8 (described later). The coating layer permits smooth insertion into and removal from the catheter. The coating layer may be formed from a fluorocarbon resin (such as polytetrafluoroethylene or “Teflon”) or a hydrophilic polymer which becomes lubricous in a wet condition.

The linear body 27 may also have a coating layer on its outer surface, in a manner similar to the tube 26, to reduce friction with the internal surface 262 of the tube 26. This coating layer produces an effect similar to that described above.

As shown in FIGS. 1-3, the capturing part 11A is comprised of two branch wires (capturing wires) 4a, 4b which branch out from the forward end 271 of the linear body 27 (or branch outwardly away from the forward end of the wire body 2) and a plurality of filaments 5a, 5b, 5c each connected at their ends to the two branch wires 4a, 4b. The illustrated embodiment of the capturing part 11A includes three filaments.

Both the base end 42 of the branch wire 4a and the base end 42 of the branch wire 4b are fixed to the forward end 271 of the linear body 27. Fixing may be accomplished in any manner without specific restrictions. One way is to wind the base ends of the branch wires 4a, 4b around the forward end 271 of the linear body 27 and bond them together by brazing, welding, or adhesion.

The three filaments 5a, 5b, 5c extend from the forward end 41 of the branch wire 4a to the forward end 41 of the branch wire 4b.

The filaments 5a, 5b, and 5c may be fixed to the branch wires 4a, 4b in any manner without specific restrictions. The method for fixing may include brazing, welding, and adhesion. The branch wires 4a, 4b may be a stranded wire composed of a plurality of linear bodies. In this case, the filaments 5a, 5b, 5c may be fixed to the branch wires 4a, 4b by tangling the filaments with the stranded wire of the branch wires. An alternative way is that the individual linear bodies of the branch wires 4a, 4b are extended so that the extended parts function as the filaments 5a, 5b, 5c, with the branch wires 4a, 4b and the filaments 5a, 5b, 5c thus being made as one body or unit. This method is relatively simple and achieves a strong connection between the filaments 5a, 5b, 5c and the branch wires 4a, 4b.

The filaments 5a, 5b, 5c are fixed to the forward end 41 of the branch wire 4a and the forward end 41 of the branch wire 4b in such a way that the central parts of the filaments 5a, 5b, 5c expand forward. In other words, the branch wires 4a, 4b and the filaments 5a, 5b, 5c form an ellipse as shown in the plan view of FIG. 1. In this embodiment, the forward end of the filaments 5a, 5b, 5c smoothly curve so that they do not damage the internal wall of the vessel 100. This configuration helps ensure higher safety.

As shown in FIG. 3, the filament 5b is in the plane (perpendicular to the paper of FIG. 2) through which the extension of the central axis of the wire body 2 (the linear body 27) passes. In other words, the filament 5b overlaps with or is coincident with the extension of the central axis of the wire body 2 in FIG. 2 (which is a side view). The filaments 5a, 5c are inclined such that they extend farther away from the extension of the central axis of the wire body 2 as they extend in the forward direction. In other words, the filament 5a slopes up and to the left with reference to the FIG. 3 illustration, while the filament 5c slopes down and to the left with reference to the FIG. 3 illustration. In addition, as seen in FIGS. 1 and 2, the tip ends or tops 51 of the filaments 5a, 5b, 5c are spaced apart from each other.

A foreign body capturing space 3 in which the embolus 200 is to be captured is formed in the capturing part 11A. The foreign body capturing space 3 is surrounded by the branch wires 4a, 4b and the filaments 5a, 5b, 5c as generally illustrated in FIGS. 1 and 2. In other words, the filaments 5a, 5b, 5c form the foreign body capturing part which assumes a basket-like shape.

The capturing part 11A constructed in this way permits the foreign body capturing space 3 to capture (hold) the embolus 200 from either the upper side or the lower side (shown in FIG. 2). This facilitates the capturing of the embolus 200.

The capturing part 11A constructed in this way is not specifically restricted in the outside diameter of the branch wires 4a 4b. The outside diameter should preferably be 0.05 to 0.9 mm, more preferably 0.1 to 0.5 mm. In the case of the branch wires 4a, 4b being formed as stranded wires, the outside diameter of the branch wires 4a, 4b denotes the overall outside diameter of the branch wires.

The filaments 5a, 5b, 5c are not specifically restricted in outside diameter. The outside diameter should preferably be 0.025 to 0.2 mm, more preferably 0.05 to 0.1 mm.

According to the present invention, the size of the capturing part 11A is not specifically restricted. It varies depending on the thickness of the vessel to be treated. The overall length (indicated by L in FIG. 1) of the capturing part 11A in its expanded state should preferably be 2 to 40 mm, more preferably 4 to 20 mm. The overall width (indicated by W in FIG. 1) of the capturing part 11A in its expanded state should preferably be 1 to 30 mm, more preferably 2 to 5 mm.

For use in the cerebral artery end (M1 portion), the capturing part 11A should have an overall length (indicated by L in FIG. 1) of at least 7 mm, preferably 8 to 15 mm, and an outside diameter (indicated by W in FIG. 1) of 2 to 5 mm, in its expanded state. This is because the inside diameter of the vessel in the cerebral artery end (M1 portion), where brain infarction occurs frequently, is about 3 to 4 mm and, according to doctors' experience, the size of thrombus causing embolus is about 3 mm in outside diameter and about 7 mm in length in many cases.

The capturing part 11A is capable of decreasing in diameter so that it can pass through (or can be inserted into) the lumen of the catheter 8. According to the present invention, the capturing part 11A is comparatively simple in structure so that it can be made thin or small relatively easily.

If the wire for removing an intravascular foreign body is to be inserted into a microcatheter, the outside diameter (indicated by W in FIG. 1) of the capturing part 11A in its contracted state should preferably be no larger than 0.53 mm (0.021 inch), more preferably no larger than 0.46 mm (0.018 inch).

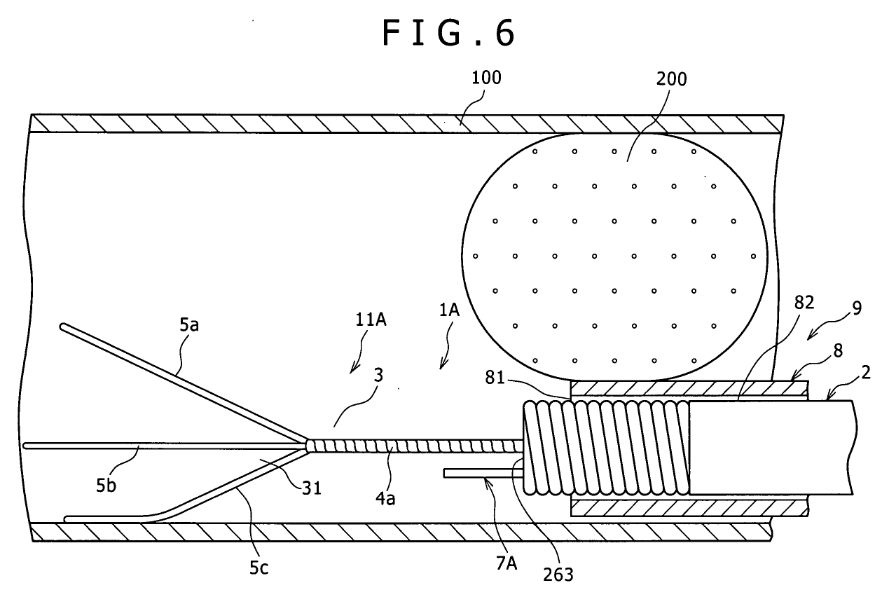

The capturing part 11A returns to its expanded state from its contracted state by its own resilience as it is pushed out (or exposed) from the forward opening 81 of the catheter 8. This takes place when the wire 1A for removing an intravascular foreign body is advanced, or moved relative to the catheter, as generally shown in FIG. 6.

Thus, the capturing part 11A changes its shape or state (expanded state and contracted state) automatically as it is pushed out from and pulled back into the forward opening 81 of the catheter 8.

The capturing part 11A, including the branch wires 4a, 4b and the filaments 5a, 5b, 5c, should preferably be formed from any radiopaque material. The specific radiopaque material is not specifically restricted. Possible materials includes, for example, gold, platinum, platinum-iridium alloy, tungsten, tantalum, palladium, lead, silver, and their alloys and compounds. Such radiaopaque materials facilitate manipulation of the capturing part to capture the embolus 200 by the capturing part 11A with the help of X-ray radioscopy and the like.

The capturing part 11A may be formed from various metallic materials and plastic (mono- or multi-) filaments. Metallic materials include stainless steel (such as SUS 304), β-titanium steel, Co—Cr alloy, piano wire, platinum-iridium alloy (Pt_{π}—Ir₁₀, Pt₈₀—Ir₂₀), noble metal alloy, nickel-titanium alloy, and other alloys having springy properties.

Of these metallic materials for the capturing part 11A, the one which exhibits superelasticity in the living organism is preferable. It reliably changes from the contracted state to the expanded state, and vice versa.

An alloy that exhibits superelasticity in the living organism refers to materials which restore nearly to their original shape after deformation (bending, stretching, and compressing) to an extent at which ordinary metals undergo plastic deformation at the living body temperature (about 37° C.). It includes so-called shape memory alloy and superelastic alloy.

The shape memory alloy and superelastic alloy are not specifically restricted. Titanium alloys (Ti—Ni, Ti—Pd, Ti—Nb—Sn) and copper alloys are preferable. The titanium alloys should preferably be composed of 30-52 atom % of titanium, with the remainder being nickel and optional elements (more than 10 atom %). The optional elements, which are not specifically restricted, may be selected from iron, cobalt, platinum, palladium, and chromium (each less than 3 atom %), and copper and vanadium (each less than 10 atom %).

Preferred superelastic alloys are those which undergo transformation from the austenite phase at normal temperature or body temperature (about 37° C.) into the martensite phase under stress.

The surface of the capturing part 11A should be provided with a means for preventing the embolus 200 (which has been captured) from slipping off from the capturing part 11A. Such an anti-slipping means increases friction between the capturing part 11A and the embolus 200, thereby allowing the capturing part 11A to reliably hold (capture) the embolus 200.

The anti-slipping means is not specifically restricted. It may be formed by coating with an elastic material (such as rubber) having a comparatively high coefficient of friction or by sand blasting which produces fine rough surfaces or surface irregularities.

Another anti-slipping means may be fiber winding or flocking, which increases the surface area of the capturing part 11A and hence increases the area which comes into contact with the embolus 200. In this way, it is possible to increase friction between the capturing part 11A and the embolus 200, as in the case of the above-mentioned examples of anti-slipping means, thereby ensuring the capturing of the embolus 200.

As shown in FIG. 1, the auxiliary wire 7A is a nearly straight linear body or member. It is fixed at the forward end portion of the inside 262 defining the internal space 261 of the tube 26(or the inside of the coil 266) such that it is approximately parallel to the lengthwise direction of the tube 26. No specific restrictions are imposed on the method for fixing the auxiliary wire 7A to the tube 26. Fixing may be accomplished by welding, brazing or adhesion. Also, the auxiliary wire 7A may be a solid wire or a hollow wire.

The auxiliary wire 7A has a forward end 71 projecting from the forward opening 263 of the tube 26 toward the capturing part 11A. In other words, the auxiliary wire 7A is closer to the base end than the capturing part 11A.

As mentioned above, the auxiliary wire 7A located in the tube 26 (or the wire 1A for removing an intravascular foreign body) permits the capturing part 11A to easily approach, or move in the direction toward, the auxiliary wire 7A as the manipulating part 273 is pulled in the direction indicated by the arrow in FIG. 1.

The auxiliary wire 7A may be formed from the same superelastic alloy as noted above for the capturing part 11A. Thus, the auxiliary wire 7A may deform under external force and restore its shape (straight) in the absence of external force. The surface of the auxiliary wire 7A may be provided with an anti-slipping means as mentioned above for the capturing part 11A. The anti-slipping means will help reliably prevent the captured embolus 200 from slipping off from the auxiliary wire 7A in much the same way as described above in connection with the capturing part 11A.

The wire 1A for removing an intravascular foreign body, which is constructed as mentioned above, permits the capturing part 11A to move relative to the auxiliary wire 7A (move toward or move away from the auxiliary wire 7A) as the manipulating part 273 is pulled away from the base end of the tube 26 in the direction indicated by the arrow in FIG. 1. Thus, the auxiliary wire 7A assumes a first state in which it projects into the foreign body capturing space 3 or the capturing part 11A as illustrated in FIG. 2 and a second state in which it retreats from the foreign body capturing space 3 as depicted in FIG. 1. In other words, the auxiliary wire 7A freely moves into in or away from the foreign body capturing space 3. The tip end (free end) of the auxiliary wire 7A is thus located closer to the tip ends of the filaments 5a, 5b, 5c when the auxiliary wire 7A is in the first state shown in FIG. 2 than when the auxiliary wire 7A is in the second state shown in FIG. 1.

The embolus 200 which has been captured in the foreign body capturing space 3 while the auxiliary wire 7A is in the second state is stabbed or otherwise held by the auxiliary wire 7A when the auxiliary wire is in the first state as shown in FIG. 1. In this way, it is possible to prevent the embolus 200 from slipping off from, or being displaced out of the foreign body capturing space 3, and hence it is possible to reliably capture the embolus 200. Eventually, it is possible to reliably remove the embolus 200 from the vessel 100.

As shown in FIG. 3, the auxiliary wire 7A may be appropriately bent at its middle or intermediate portion so that its forward end 71′ approximately coincides with the central axis of the wire body 2. This alternative version of the auxiliary wire 7A is illustrated by a broken line in FIG. 3.

As shown in FIG. 2, the regulating means 28 regulates the maximum length over which the auxiliary wire 7A in the first state projects into the foreign body capturing space 3. In the illustrated embodiment, the regulating means 28 comprises a projecting part 267 and an expanded part 274. The projecting part 267 projects in the radial direction from the inside 262 of the tube 26 in the neighborhood of the base opening 265 of the tube 26. The expanded part 274 is a part which expands in the radial direction from the outside of the linear body 27 and is positioned in the internal space 261 of the tube 26. The expanded part 274 can be formed by fixing a ring-shaped member to the linear body 27.

The projecting part 267 of the tube 26 and the expanded part 274 of the linear body 27 are constructed such that the face 267a at the forward end side of the projecting part 267 comes into contact with the face 274a at the base end side of the expanded part 274 when the auxiliary wire 7A assumes the first state (or when the linear body 27 moves toward the base end relative to the tube 26). This prevents the linear body 27 from moving excessively toward the base end. When the projecting part 267 and the expanded part 274 are in contact with each other, the distance indicated by the length M in FIG. 2 represents the maximum length of projection of the auxiliary wire 7A.

The regulating means 28 constructed as mentioned above protects the embolus 200 from being stabbed excessively by the auxiliary wire 7A. In this way, it is possible to prevent the embolus 200 from being broken by the auxiliary wire 7A.

It is to be understood that the maximum length M of projection of the auxiliary wire 7A is not specifically restricted. It should preferably be shorter than the overall length of the capturing part 11A in its expanded state, but it depends on the length L of the capturing part 11A. It should preferably be 2 to 20 mm, more preferably 3 to 6 mm.

The expanded part 274 may be fixed to the linear body 27, with its position properly adjusted. This adjustment adjusts the maximum length M of projection. In this way, it is possible to set up the maximum length M of projection according to the size (length) of the embolus 200.

The number of the branch wires forming a part of the capturing part 11A is not limited to two; and may include three or more branch wires. Also, the number of filaments is not limited to three; and may include two, four or more filaments. Further, although the embodiment described above include one auxiliary wire 7A, the invention is not limited in that regard and may include two or more auxiliary wires.

A medical instrument 9 according to the present invention includes the wire 1A for removing an intravascular foreign body and the catheter 8 which has the lumen 82 formed therein.

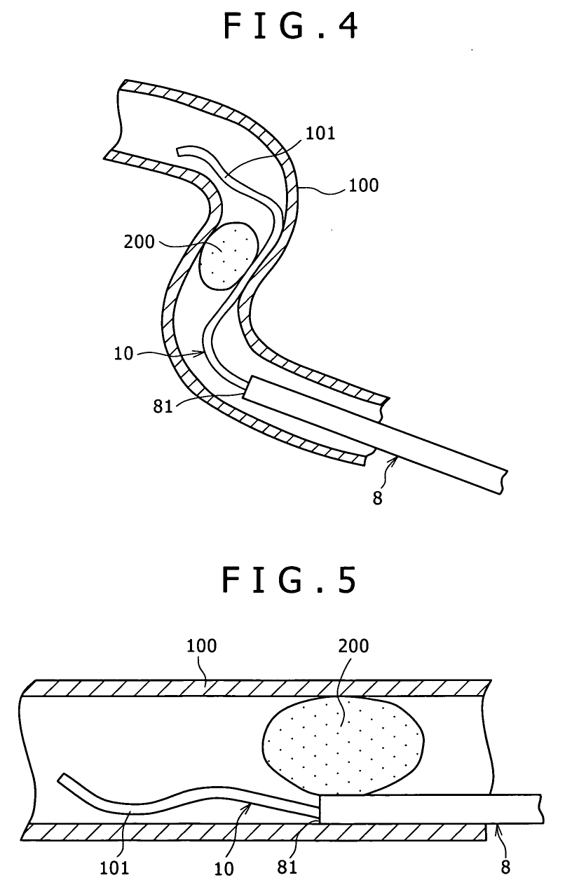

Set forth below is a detailed description of one way of using the wire 1A for removing an intravascular foreign body.

[1] FIG. 4 depicts a vessel 100 clogged with the embolus 200 (such as thrombus) and inhibiting blood flow. The embolus 200 is almost immobile because it is pushed against the inner wall of the vessel 100 by blood pressure. It is assumed that the presence of the embolus 200 is previously confirmed by radioscopy.

The first step is to insert the catheter (microcatheter) 8 and the guide wire 10 (which has been passed through the lumen 82 of the catheter 8) into the vessel 100. The second step is to project the guide wire 10 from the forward open end 81 of the catheter 8 beyond the embolus 200. In other words, the second step is carried out such that the forward end 101 of the guide wire 10 passes through the gap between the embolus 200 and the inner wall of the vessel 100 and moves beyond the embolus 200. This operation can be accomplished by using the guide wire 10 (micro-guide wire) which has good lubricity.

[2] After the forward end 101 of the guide wire 10 has moved past the embolus 200, the catheter 8 is advanced relative to the guide wire 10, so that the forward end of the catheter 8 is positioned in the gap between the embolus 200 and the inner wall of the vessel 100 as shown in FIG. 5. This operation can be relatively easily performed because the forward end of the catheter relatively smoothly moves in the gap by moving along the guide wire 10.

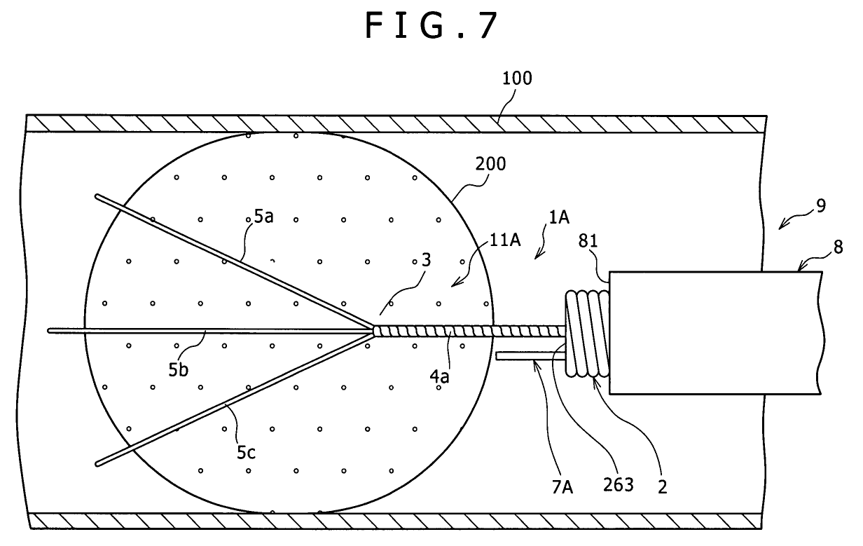

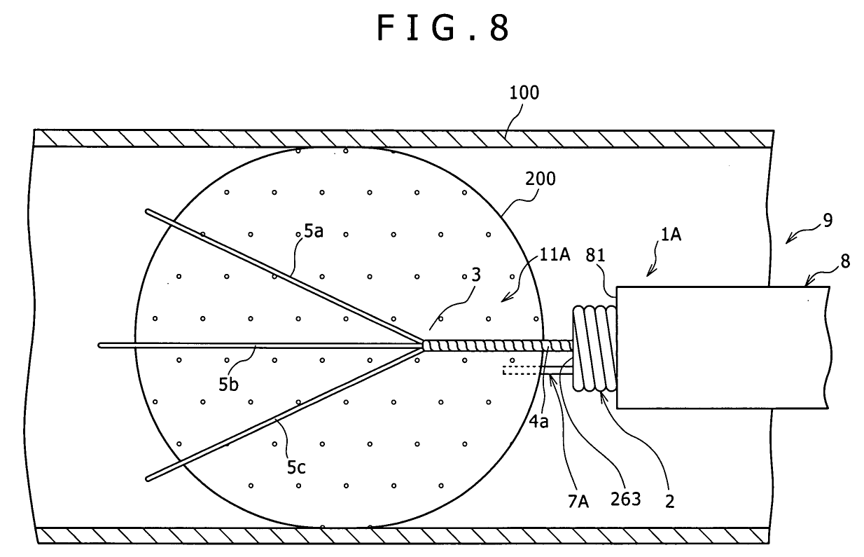

According to the conventional therapy, the above-mentioned stage is followed by injection of a thrombolytic agent through the catheter 8. However, doctors often experience that there are emboli 200 which are not dissolved by a thrombolytic agent or dissolution of the emboli 200 by a thrombolytic agent takes a long time. The present invention is effective in such a case.

[3] The step shown in FIG. 5 is followed by the next step in which the guide wire 10 is removed and the wire 1A for removing an intravascular foreign body is inserted into the lumen 82 of the catheter 8.

[4] As shown in FIG. 6, this is accomplished so that the forward end of the coil 266 of the wire body 2 projects from the forward open end 81 of the catheter 8. As the result, the capturing part 11A of the wire, which has been positioned in the catheter 8 in its contracted shape or state, expands to form the foreign body capturing space 3 in which the embolus 200 is to be captured.

[5] Then, from the state shown in FIG. 6, the catheter 8 is slightly moved toward the base end so that the forward end of the catheter 8 retreats to the back end of the embolus 200. Now, the embolus 200 is captured (scooped) by the foreign, body capturing space 3 of the capturing part 11A as shown in FIG. 7. In other words, the embolus 200 enters the foreign body capturing space 3 from the upper side shown in FIGS. 6 and 7. At this time, the embolus 200 is tightened up by the capturing part 11A.

[6] From the state shown in FIG. 7, the capturing part 11A is moved toward the base end by manipulating the manipulating member 273 of the linear body 27 so that the embolus 200 (or the capturing part 11A) approaches the auxiliary wire 7A. The capturing part 11A is moved further toward the base end so that the auxiliary wire 7A contacts or stabs the embolus 200 as shown in FIG. 8.

[7] Then, the wire 1A for removing an intravascular foreign body and the catheter 8 are removed all together, with the state shown in FIG. 8 being maintained. Thus, the embolus 200 is collected in the guiding catheter or sheath introducer.

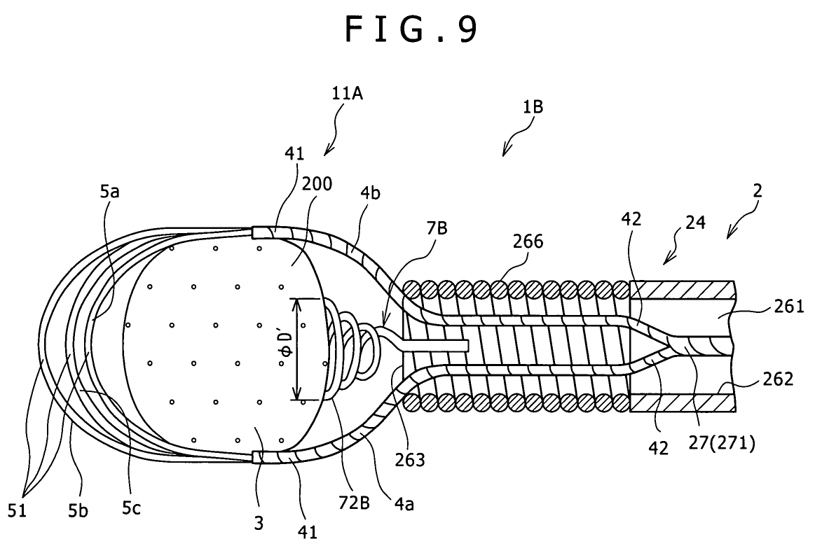

FIG. 9 is a plan view, partially in longitudinal cross-section, illustrating a portion of the wire for removing an intravascular foreign body (in the first state) according to a second embodiment of the present invention. In the following description, the right side and left side of FIG. 9 are designated as “base end (proximal end)” and “forward end (distal end)”, respectively.

The description below describes aspects of the wire for removing an intravascular foreign body according to the second embodiment that differ from aspects of the embodiment described above. A detailed description of features of the wire according to the second embodiment that are similar to features already described above in will not be repeated.

The wire for removing an intravascular foreign body according to the second embodiment is virtually identical to the embodiment described above, except for the shape of the auxiliary wire. As shown in FIG. 9, the auxiliary wire 7B in this second embodiment of the wire 1B for removing an intravascular foreign body has an expanded part 72B which comes into contact with the embolus 200 in when the wire is in its first state. The expanded part 72B has a helical shape, with the loop diameter of each successive loop of the helical shape increasing toward the forward end. The auxiliary wire 7B formed in this manner permits the embolus 200 to be held (or pressed down) between the expanded part 72B and the filaments 5a, 5b, 5c. In this way, it is possible to reliably capture and remove the embolus 200 in the vessel 100.

By virtue of its helical shape, the expanded part 72B holds the embolus, 200 with a relatively mild force to lessen the chance of, and preferably avoid, the possibility of breaking the embolus 200. The outside diameter D′ (φD′ in FIG. 9) at the forward end of the expanded part 72B is not specifically restricted. It should preferably be 1 to 3 mm, and it should preferably be smaller by about 1 mm than the outside diameter (W described above) when the capturing part 11A is expanded.

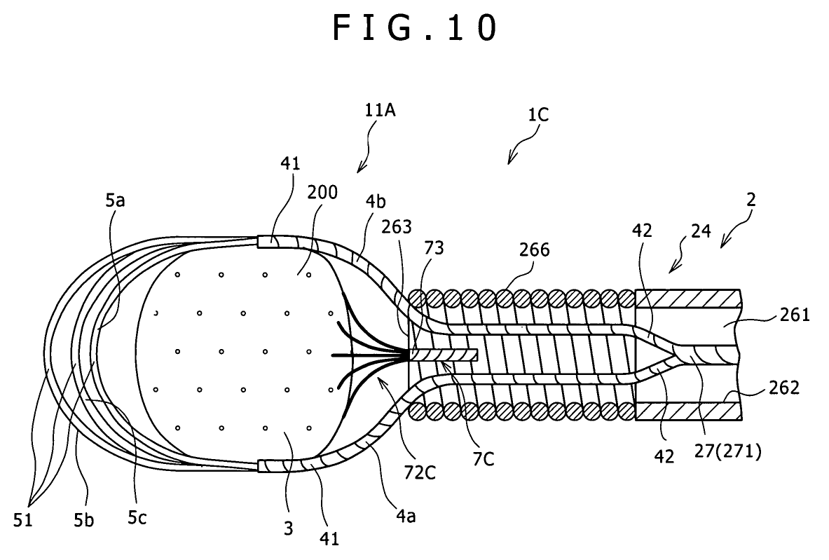

FIG. 10 is a plan view, partially in longitudinal cross-section, of a portion of the wire for removing an intravascular foreign body (in the first state) according to a third embodiment of the present invention. In the description which follows, the right side and left side of FIG. 10 are designated as “base end (proximal end)” and “forward end (distal end)”, respectively.

The description below describes aspects of the wire for removing an intravascular foreign body according to the third embodiment that differ from aspects of the embodiments described above. A detailed description of features of the wire according to the third embodiment that are similar to features already described above in will not be repeated.

The wire for removing an intravascular foreign body according to the third embodiment is virtually identical to the embodiments described above, except for the shape of the auxiliary wire. As shown in FIG. 10, the auxiliary wire 7C of the wire 1C for removing an intravascular foreign body has an expanded part 72C, which comes into contact with the embolus 200 when the auxiliary wire 7C is in its first state. The expanded part 72C comprises a plurality of short linear bodies radially projecting toward the forward end from the vicinity of the intermediate part 73. In the illustrated embodiment, the expanded part 72C comprises eight short linear bodies.

The auxiliary wire 7C formed in this manner permits the embolus 200 to be held (or pressed down) between the expanded part 72C (i.e., the forward ends of short linear bodies) and the filaments 5a, 5b, 5c when the auxiliary wire 7C is in the first state. In this way, it is possible to reliably capture and remove the embolus 200 in the vessel 100.

The auxiliary wire 7C may be constructed such that four short linear bodies at the expanded part 72C stab or contact the embolus 200.

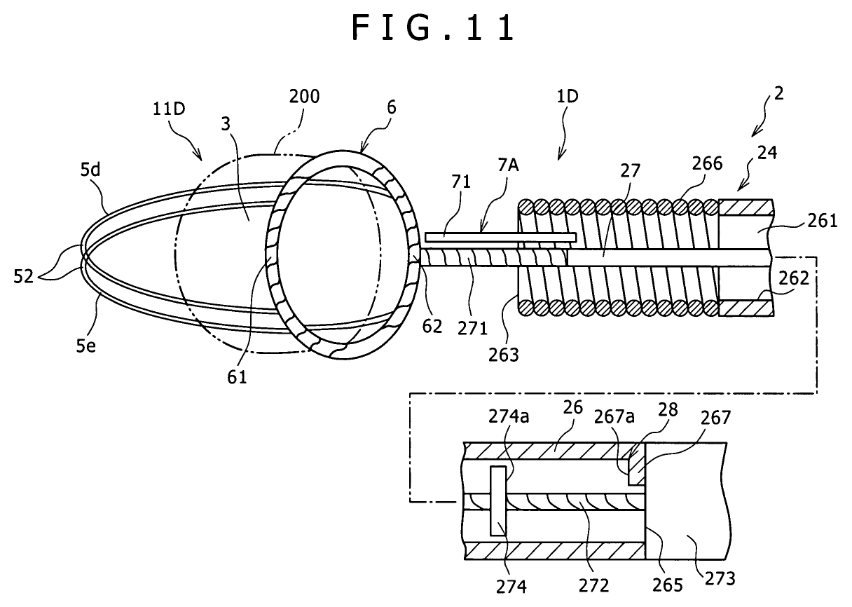

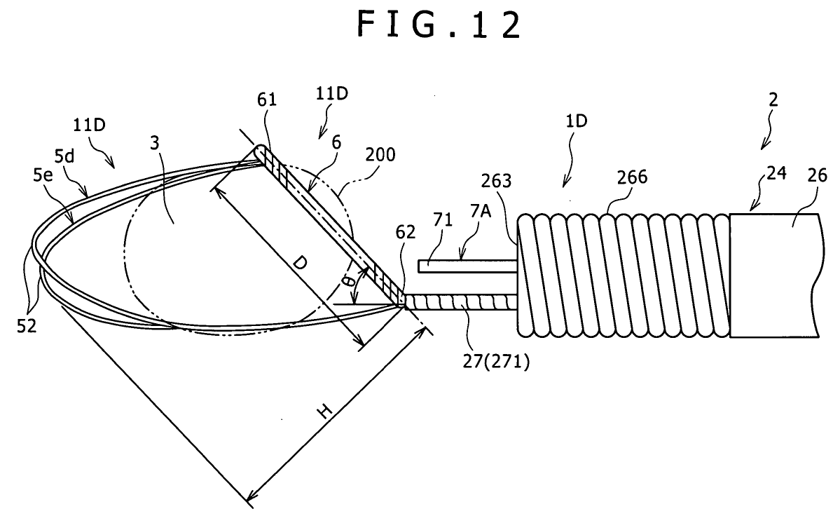

FIG. 11 is a plan view, partially in longitudinal cross-section, of a portion of the wire for removing an intravascular foreign body (in the first state) according to a fourth embodiment of the present invention. FIG. 12 is a side view of the wire illustrated in FIG. 11 for removing an intravascular foreign body. In the following description the right side and left side of FIGS. 11 and 12 are designated as “base end (proximal end)” and “forward end (distal end)”, respectively.

The wire for removing an intravascular foreign body according to the fourth embodiment is identical with that in the first embodiment except for the construction of the capturing part.

As shown in FIGS. 11 and 12, the capturing part 11D of the wire 1D for removing an intravascular foreign body includes capturing wires which include a loop wire 6 and several filaments or wires 5d, 5e. The loop wire 6 is arranged on the linear body 27 of the wire body (the forward end of the wire body 2). The filaments (two in this embodiment) are twisted with the loop wire 6.

As shown in FIG. 12, the loop wire 6 is bent or angled relative to the axis of the wire body 2 so that the plane containing the loop wire 6 forms an angle other than zero degrees with respect to the axis of the wire body 2.

The method for fixing the loop wire 6 to the linear body 27 (wire body 2) is not specifically restricted. Fixing may be accomplished by winding or braiding the base end 62 of the loop wire 6 to the forward end 271 of the linear body 27 and then performing brazing, welding, or adhesion on them.

As shown in FIG. 12, the loop wire 6 in its natural state stands or is inclined with respect to the linear body 27 (the wire body 2). This state is referred to as the “standing state.” The angle of the loop wire 6 (θ in FIG. 12) with respect to the central axis of the linear body 27 should preferably be about 20 degrees to 90 degrees, more preferably 40 degrees to 60 degrees. This construction permits the forward end 61 of the loop wire 6 to move into the gap between the embolus 200 and the inner wall of the vessel 100 when the embolus 200 is to be captured. In this way it is possible to reliably capture the embolus 200.

In the standing state, the loop diameter (D in FIG. 12) of the loop wire 6 is not specifically restricted; but should preferably be 1 to 20 mm, more preferably 2 to 5 mm.

As shown in FIG. 11, the filaments 5d, 5e are attached to different parts of the loop wire 6 which are arranged symmetrically.

The filaments 5d, 5e are forwardly curved like an arch, and they cross each other at the tops 52 of their arches. Crossing means that the filaments 5d, 5e are close to each other, but does not necessarily mean that they are in contact with each other. They may be in contact with each other such that they move relative to each other.

In the standing state, the distance between the loop wire 6 and the top 52 (the distance H in FIG. 12) is not specifically restricted. It should preferably be 3 to mm, more preferably 3 to 10 mm.

The capturing part 11D constructed in this manner can reliably capture and permit removal of the embolus 200.

It is to be understood that the number of filaments is not limited to two; as it may be three or more.

The foregoing description describes various embodiments of a wire for removing an intravascular foreign body and a medical instrument utilizing such a wire. However, it is to be understood that features and aspects of the wire and medical instrument may be modified or replaced by (or reinforced with) with features and aspects that function in a similar manner.

For example, two or more of the above-described embodiments, or features from such embodiments, may be combined to complete the wire for removing an intravascular foreign body.

The first embodiment may be modified to employ the auxiliary wire used in the second or third embodiment, while the fourth embodiment may be modified to employ the auxiliary wire used in the second or third embodiment.

Also, the wire for removing an intravascular foreign body may be modified such that the auxiliary wire is moved relative to the capturing part rather than the capturing part being moved relative to the auxiliary wire.

In the third embodiment described above, the expanded part of the auxiliary wire is contractable and expandable; but it may be constructed such that the expanded part is expandable when the auxiliary wire is in the first state.

The operation of the linear body of the wire proper is not limited to manipulation by the manipulating part. The operation can be accomplished in other ways, such as by driving a rack and a pinion attached to the tube of the wire proper and the linear body, respectively. In this way, it is possible to easily manipulate the linear body.

The principles, preferred embodiments and manners of use of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A wire for removing an intravascular foreign body, comprising:
a flexible elongated wire body;
a capturing part arranged at a forward end of the wire body;
a space formed in the capturing part to capture an intravascular foreign body; and
an auxiliary wire arranged at the forward end of the wire body, the auxiliary wire being movable relative to the capturing part to position the auxiliary wire in a first state in which the auxiliary wire projects into the space and a second state in which the auxiliary wire is retreated from the first state.

2. The wire for removing an intravascular foreign body according to claim 1, wherein the wire body comprises a tube possessing a forward end and a linear body passing through the tube, the linear body possessing a forward end, the auxiliary wire being arranged at the forward end of the tube and the capturing part being arranged at the forward end of the linear body.

3. The wire for removing an intravascular foreign body according to claim 1, wherein the auxiliary wire is closer to a base end of the wire body than the capturing part.

4. The wire for removing an intravascular foreign body according to claim 1, wherein the auxiliary wire in the first state is adapted to be in contact with the foreign body and has an expanded part which expands toward the forward end.

5. The wire for removing an intravascular foreign body according to claim 4, wherein the expanded part possesses a helical shape.

6. The wire for removing an intravascular foreign body according to claim 5, wherein the helical shape of the expanded part gradually increases in outer dimension toward a forward end of the expanded part.

7. The wire for removing an intravascular foreign body according to claim 1, further comprising means for limiting a maximum extent to which the auxiliary wire projects into the space in the first state.

8. A medical instrument comprising:
the wire for removing an intravascular foreign body according to claim 1; and
a catheter having a lumen that receives the wire for removing an intravascular foreign body.

9. A wire for removing an intravascular foreign body, comprising:
a flexible wire tube possessing an outer diameter which permits the flexible wire tube to be positioned in a blood vessel of a living body;
a plurality of capturing wires extending forward of a forward end of the flexible wire tube and together defining a space of a size to capture an intravascular foreign body; and
an auxiliary wire, the auxiliary wire and the plurality of capturing wires being movable relative to one another to position the auxiliary wire in one position in which the auxiliary wire projects forward of the forward end of the flexible wire tube into the space to engage an intravascular foreign body captured in the space and another position in which the auxiliary wire is retracted relative to the one position in a direction away from the space.

10. The wire for removing an intravascular foreign body according to claim 9, wherein the auxiliary wire possesses a linear shape.

11. The wire for removing an intravascular foreign body according to claim 9, wherein the auxiliary wire possesses a helical shape.

12. The wire for removing an intravascular foreign body according to claim 9, wherein the auxiliary wire possesses a helical shape that gradually increases in outer dimension in a direction away from the flexible wire tube.

13. The wire for removing an intravascular foreign body according to claim 9, wherein the auxiliary wire is comprised of a plurality of linear bodies arranged to expand radially outwardly in a direction away from the flexible wire tube.

14. The wire for removing an intravascular foreign body according to claim 9, wherein the auxiliary wire is fixed to the flexible wire tube to move together with the flexible wire tube.

15. The wire for removing an intravascular foreign body according to claim 14, wherein the plurality of capturing wires are fixed to an elongated linear body so that the plurality of capturing wires and the elongated linear body move together as a unit, a portion of the linear body being positioned in a hollow interior of the flexible wire tube, the auxiliary wire and the plurality of capturing wires being movable relative to one another by moving the elongated linear body and the flexible wire tube relative to one another.

16. The wire for removing an intravascular foreign body according to claim 15, wherein the elongated linear body includes a portion which contacts a part of the flexible wire tube when the elongated linear body and the flexible wire tube are moved relative to one another to position the auxiliary wire in the one position to limit a maximum extent to which the auxiliary wire projects into the space.

17. The wire for removing an intravascular foreign body according to claim 9, wherein the plurality of capturing wires are fixed to an elongated linear body so that the plurality of capturing wires and the elongated linear body move together as a unit, a portion of the linear body being positioned in a hollow interior of the flexible wire tube.

18. A method of removing an intravascular foreign body from a blood vessel of a living body, comprising:
introducing an intravascular foreign body removing wire in a blood vessel of a living body, the intravascular foreign body removing wire comprising a flexible elongated wire body, a capturing part positioned forward of a forward end of the wire body, and an auxiliary wire;
capturing an intravascular foreign body within a space of the capturing part;
initiating relative movement between the capturing part and the auxiliary wire to move the auxiliary wire into engagement with the intravascular foreign body captured within the space of the capturing part; and
withdrawing the intravascular foreign body removing wire from the blood vessel with the intravascular foreign body captured within the space of the capturing part.

19. The method according to claim 18, wherein the intravascular foreign body removing wire is introduced into the blood vessel through a catheter.

20. The method according to claim 18, wherein relative movement is initiated between the capturing part and the auxiliary wire by moving the capturing part relative to the auxiliary wire, the capturing part being moved relative to the auxiliary wire until a part fixed to and movable with the capturing part contacts the elongated wire body to prevent further relative movement between the capturing part and the auxiliary wire.
